(19) United States
(12) Patent Application Publication
(10) Pub. No.: US 2009/0292321 A1
(43) Pub. Date: Nov. 26, 2009

(54) BONE ANCHORING DEVICE

(76) Inventor: Michel Collette, Bruxelles (BE)

Correspondence Address: YOUNG & THOMPSON, 209 Madison Street, Suite 500, ALEXANDRIA, VA, 22314, US

(21) Appl. No.: 12/306,475

(22) PCT Filed: Jun. 25, 2007
(86) PCT No.: PCT/EP2007/005567

§ 371 Date: Dec. 23, 2008

(30) Foreign Application Priority Data
Jun. 23, 2006 (BE) 2006/0348

(51) Int. Cl. A61B 17/86; A61B 17/04

(52) U.S. Cl. 606/303; 606/232

(57) ABSTRACT

The invention concerns an anchoring system for fixing a ligament graft in a bone tunnel. The invention concerns a hollow socket (1) to be anchored in the bone tunnel for passing through relay bands or suture. The socket has a cylindrical outer wall (6), a cylindrical or tapered inner wall (6′) and two orifices (11, 12), the inner wall being capable of enclosing and locking the bands via the action of a locking member inserted in the socket. The outer wall (6) is provided with means to be secured to the bone tunnel (32), for example a screw thread fitted to the bone anchoring device.

This invention relates to an anchoring system for securing a ligament graft in a bone tunnel.

All of the sports that involve twisting motions, such as rugby, soccer, skiing, etc., bring about a significant tension of the knee ligaments and therefore comprise a significant risk of traumatic injury. This risk is considerably increased when the sports are practiced at high levels.

The short and thick anterior cruciate ligament extends obliquely from the prespinal surface of the upper face of the tibia to the axial face of the external condyle of the femur and ensures the rotary anterior stability of the knee.

The accidental rupture of the anterior cruciate ligament constitutes one of the most common injuries in sports pathology of the knee, often forcing the injured individual to give up sports partially or even completely.

Nevertheless, there are surgical techniques for reconstruction of the anterior cruciate ligament that make it possible to restore the stability of the knee and, consequently, its functional capacities.

The reconstruction of an anterior cruciate ligament can be performed by means of a ligament graft introduced into bone tunnels, tibial and femoral, whose articular openings coincide with the zones for inserting the natural anterior cruciate ligament.

The definitive anchoring of the graft is achieved by incorporation and gradual adhesion of the graft to the walls of the bone tunnel.

This incorporation takes place relatively quickly (about six to eight weeks) if a graft taken from the patellar tendon, comprising a small bone block from the kneecap and tibia at each of its ends, is used. This bone-tendon-bone type graft thus comprises a central ligament part and two bone parts, the latter allowing very good attachment in the bone tunnel.

This kind of sampling comprises significant potential drawbacks, namely embrittlement of the extensor apparatus, chronic pain, and the risk of fracturing the kneecap or of rupturing the patellar tendon when they have been embrittled by the sampling to which they were subjected.

To avoid these drawbacks, it is possible to employ ligament grafts taken from the inner hamstring tendons, i.e., where the terminal tendons of the sartorium, gracilis, and semitendinous muscles meet, called a DI-DT-type graft.

It actually involves a less-invasive technique, whose risk of undesirable effects linked to sampling is much lower.

However, the graft then consists of a pure tendinous tissue, i.e., deficient in bone blocks at its ends. This relative to the bone-tendon-bone graft poses a technical problem for securing the graft in the bone tunnel.

It is necessary to know, actually, that it will take at least three months and sometimes more for the inserted tendinous tissue to adhere properly to the wall of the bone tunnel. During this entire time, the solidity of the mounting will therefore rest essentially on the quality of the artificial attachments that will be installed during the intervention.

If the attachments of the graft are not adequately effective, the repeated tension forces associated with regaining knee mobility will cause a gradual sliding of the graft in the bone tunnel with loss of the initial tension and recurrence of the laxity.

Several methods for securing the ligament grafts in a bone tunnel are known, each of them having, at various degrees, serious limitations.

A conventional method of attachment consists in inserting a so-called interference screw between the ligament graft and the wall of the bone tunnel in which the graft will have been previously inserted.

Experimentally, it was possible to show that the mechanical tearing resistance of DI-DT-type grafts that are attached by interference screws is on the order of 35 to 40 daN on average.

In some extreme cases, the mechanical tearing resistance cannot exceed 20 daN.

In addition, the most recent experimental studies that examine the behavior of these grafts when they are subjected to cyclic tension forces so as to simulate what will be produced during rehabilitation show that this type of attachment does not make it possible to effectively neutralize the gradual sliding of the graft that takes place during each tension peak.

This sliding produces a gradual loss of the initial tension and can even, after several hundreds of cycles, cause the complete tearing of the graft outside of the bone tunnel.

Finally, the crushing of the tendinous tissue by the screw, all the more important as a solid attachment is desired, can be very harmful for the histological evolution of the tendinous tissue that is at risk of being sheared, of necrotizing, and, finally, of sometimes being incorporated very poorly into the bone.

Another device that is intended to prevent the drawbacks of the interference screws consists in passing into the ligament loop a relay strip made of synthetic material that is itself attached to a small metal bar (Endobutton type). After having traversed the entire bone tunnel along its longitudinal axis by retracting behind it the relay strip and the ligament loop, this bar pivots and is applied at the bone cortex and thus neutralizes the possible tearing of the graft.

This type of mounting makes it possible to achieve a resistance that, according to the literature, does not, however, exceed 50 daN on average.

However, it is clearly demonstrated that, subjected to cyclic traction forces, the relay strip deforms and gradually elongates permanently (all the more so since its initial length is great), which will also bring about a gradual loss of the initial tension applied to the graft during its installation.

It is assumed, however, that the movements of the knee that take place during daily life, and, consequently, during free rehabilitation exercises, will produce cyclic traction peaks in the cruciate ligament, or its substitute can achieve 50 daN.

This means that to allow the patient to undertake an intensive and premature rehabilitation, which constitutes a more and more pressing demand on the part of the sports patients, after a reconstruction of the anterior cruciate ligament, clearly comprises risks of deterioration of the mechanical properties of the graft, namely recurrent laxity or risk of accidental tearing.

These risks of degradation of the attachment in an already quite real bone tunnel in the case of use of a bone-tendon-bone-type of graft are still greater than if a DI-DT-type graft that is yet much more advantageous from the standpoint of the secondary drawbacks to the sampling is used.

One object of the invention is therefore to propose an attachment device for a ligament graft in a bone tunnel that has a high resistance to the pulling to limit the risks of tearing or sliding from the ligament graft in a bone tunnel.

The patent application WO 2004/045465 shows the closest prior art and relates to a technique for attaching a ligament graft that is suspendend by textile strips that are screwed into the bone tunnels by means of a special screw that was referred to as a TLS screw (Tape Locking Screw).

The exceptional mechanical properties of this attachment system compared to other market systems have been absolutely indisputably demonstrated in the mechanical-engineering laboratory. However, the experience attained in ongoing surgery for more than three years now, after having produced more than 300 cases of surgical reconstruction by using this attachment system, shows that several technical problems persist that can reduce the quality of the results in vivo relative to the results of the experimental mechanical tests.

These mechanical problems are linked, on the one hand, to individual variations of the bone quality and, on the other hand, to the quality of the placement of the TLS screw.

The bone quality varies considerably from one individual to the next based on age, sex, rate of mineralization, etc. As it was confirmed experimentally, the resistance to tears and primarily to the sliding of the strip depends greatly on the quality of the bone in which the screw is implanted. A tender bone will withstand clearly less than a hard bone.

On the other hand, the process of healing a traumatized bone first of all comprises a bone resorption phase due to the work of osteoclasts that necessarily precedes the secondary reconstruction phase in the work of osteoblasts. It is therefore not ruled out that the solidity of the initial mounting is significantly altered during the bone resorption phase, which could, for some time, endanger the solidity of the mounting and could run the risk by relative sliding of the strip of reducing the tension in the graft and, consequently, the stability of the knee that was restored during the operation. The resistance of the bone not being measurable case by case, such a potential weakening of the mounting would make it necessary for the surgeon to return to techniques for protecting the graft (splint, partial relief of load stress) for all of the cases on which operations are performed, whereas the essential object of the TLS technique was, by proposing an extremely solid attachment system, specifically also to be free of protection techniques without running the risk of compromising the quality of the final mechanical result.

The other possible factors for reducing the effectiveness of the system are linked to the quality of the implantation of the screw.

The radiographic monitoring (in particular by scanner) that has been carried out systematically on patients has shown that the screw does not always very faithfully follow the shaft of the bone tunnel into which it is inserted. The divergence from the path of the screw relative to the shaft of the bone tunnel into which the strips pass constitutes a frequent and unfortunately uncontrollable risk by the surgeon if this is not radiographically what in practice is not feasible during an intervention. The post-surgical monitoring makes it possible to identify the problem, but, at this time, it is obviously no longer possible to correct the situation. It is clear that a divergent screw relative to the bone tunnel will be less effective for withstanding the sliding of the strips than a screw that perfectly follows the axis of the bone tunnel. The factor unfortunately constitutes a potential pitfall inherent to the TLS technique, difficult to avoid and whose impact on the quality of the mounting varies from case to case and cannot be measured.

The third clearly identified risk factor is the depth of insertion of the screw. Since the screw is introduced by a minimum incision (approximately 1 cm), the surgeon works blind and has to trust the graduations of the placement instruments to assess the depth of insertion of the screw primarily into the femur where the thickness of the integuments is much greater than in the tibia. However, the experiment has shown that there is a significant risk of making an error in the evaluation of the depth of insertion of the screw. Too deep or too shallow a screw will not provide the same attachment quality of the strips as if it were at its proper level. This concomitant risk also constitutes a reduction factor of the mechanical quality whose importance is individual and not measurable. In addition, if a screw is not inserted deeply enough, it can cause irritation of the soft tissues at the site where it emerges from the bone and can produce a chronic inflammatory reaction at this location that is at least uncomfortable and can even be painful.

Another drawback of the original TLS technique resides in the fact that it forces the surgeon to divide a bone tunnel into two separate parts, requiring cuttings of different calibers: a wide portion (femoral and tibial spaces) ending in an intra-articular area designed to accommodate the end of the ligament graft, hollowed out in a retrograde manner, from the inside to the outside, and a fine portion, emerging toward the outside, hollowed out from the outside toward the inside, making possible the tapping from the outside to the inside to prepare the housing of the screw. Although the technical solutions for obtaining this result have been provided and described in the document of Patent WO2004/045465 (flanged augers), it is necessary to recognize that this embodiment is certainly more difficult and longer to produce than a bone tunnel of a single caliber as in the conventional ligamentoplasty techniques.

It is not technically impossible to use the TLS system with bone tunnels of a single caliber, but in practice, this leads to having to use extremely bulky screws since their housing should be prepared from a tunnel that has already been hollowed out to the caliber of the graft. However, from one patient to the next, this caliber may vary between 7 and 10 mm, whereas in the original TLS technique, the housing of the screw is prepared from a tunnel whose caliber is always 4.5 mm in diameter.

Knowing that all of these adverse factors can be combined at various degrees in the same individual, a need to enhance the TLS technique as described in the above-mentioned patent application is still felt.

The object of this invention is to propose a system that makes it possible to obtain in each case an optimum quality of securing the TLS system, i.e., in all of the patients, regardless of their bone quality, while eliminating the risks of bad positioning of the screw (divergence, too deep or too shallow).

This invention makes it possible not only to make the quality of the results uniform but in addition, owing to a simplified technique, it makes it possible to use bone tunnels of a single caliber.

This invention proposes enhancing the effect of a screw or a similar locking element by designing in the addition of a hollow sleeve that comprises an outside wall, an inside wall, and two openings: one generally, but not necessarily, wide, and the other narrower.

More specifically, the invention proposes a hollow sleeve to be anchored in a bone tunnel that is designed for the passing of relay strips or suture thread, characterized in that it has an outside wall, an inside wall, and two openings, whereby said inside wall is able to clamp and lock said strips by the effect of a locking element that is inserted into the sleeve.

Other aspects of the invention are mentioned in the dependent claims attached to this document.

The surgical technique is easily adapted for putting into practice this new device, and the invention therefore also relates to the associated process.

According to one embodiment, the inside wall of the sleeve corresponds to the reverse image of a strip locking screw. In other words, in the thickness of the inside wall of the sleeve, there is a spiral furrow whose shape corresponds exactly to the thread of the locking screw. The depth of the furrow is calculated such that after tightening the screw in its sleeve, the entire screw has penetrated the sleeve after having compacted the strips into the furrows of the sleeve according to a predetermined, optimum torque based on the mechanical properties of the material that is used.

According to an optional aspect of the invention, the outside wall of the sleeve is equipped with at least four anti-rotational ailerons that are designed to neutralize in the bone the torsion torque induced by the tightening of the screw in its sleeve.

The widest opening, directed toward the outside, corresponds to the entrance opening for penetration of the screw. Both in the femur and in the tibia, it is known that the bone tunnel to be provided forms with the cortical bone a variable angle in case by case of 30 to 60°. So as to facilitate the complete burying of the sleeve in its bony space, according to one embodiment, the entrance opening of the screw is sloped by about 30° relative to the perpendicular plane to the large axis of the sleeve. According to another aspect of the invention, in the union between this entrance opening and the outside wall (short side) of the sleeve, there is a flange that is designed to abut against the bone cortex, thus keeping the sleeve from penetrating beyond this cortex. Although the conical shape of the sleeve already constitutes a brake in itself to a possible excess of penetration, this overhang provides additional safety and primarily makes it possible to place the sleeve in a reproducible way from one individual to the next regardless of the angulation of the bone tunnel relative to the cortex.

According to another embodiment, the sleeve is not conical but cylindrical over its entire outside surface. In contrast, the inside cavity could be either conical or cylindrical according to the locking mechanism of the strip that is selected.

The invention will be better understood from examining the accompanying drawings that are presented only by way of non-limiting examples, in which:

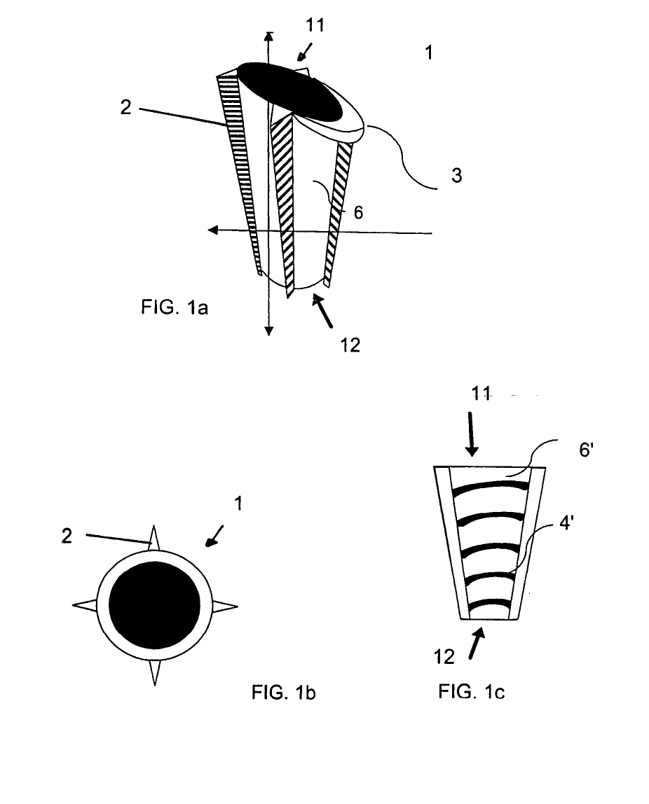

FIG. 1a shows diagrammatically and in perspective a sleeve according to the invention.

FIGS. 1b and 1c are corresponding horizontal and vertical cutaways.

FIG. 1d shows the general shape of a sleeve.

FIG. 1e shows how the sleeve 1 is positioned in a bone tunnel.

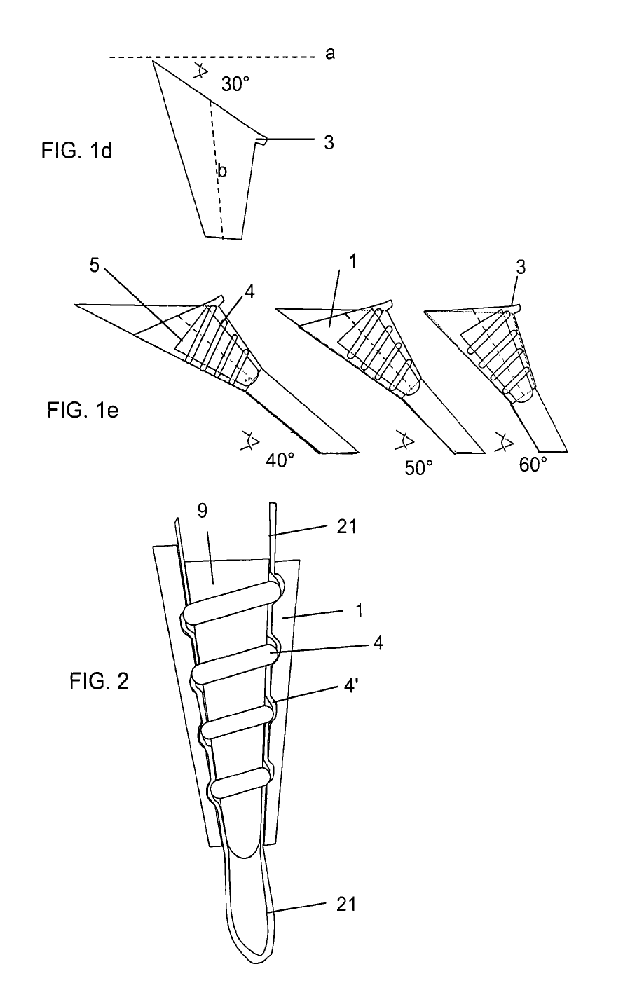

FIG. 2 shows the screw 9 in place in the sleeve 1.

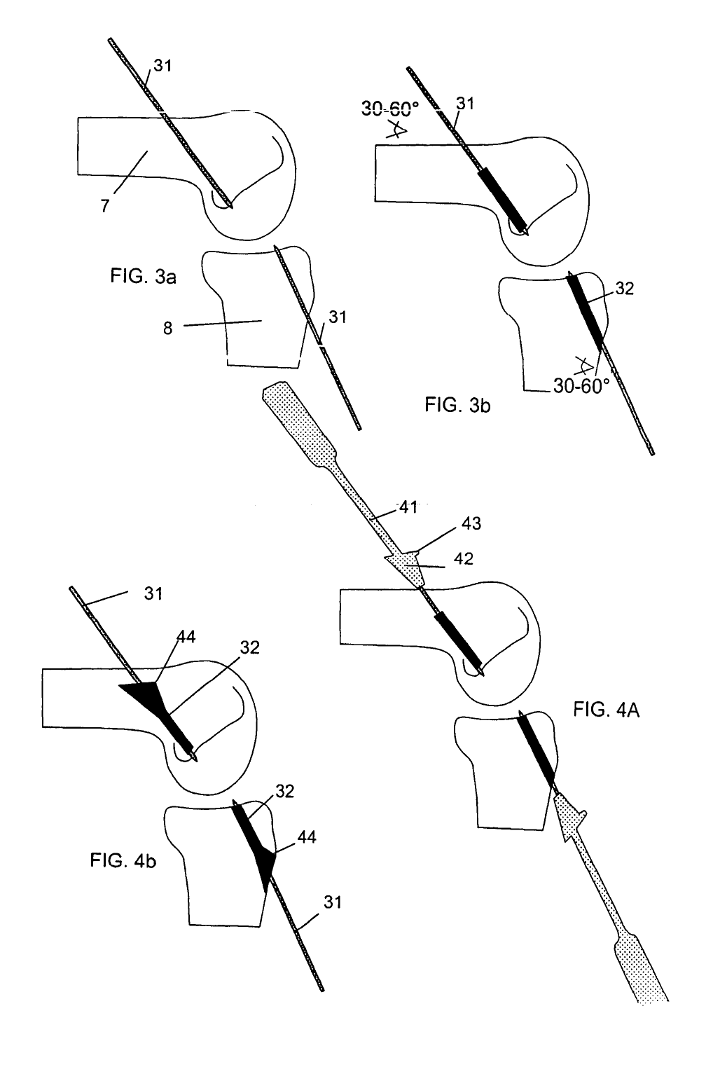

The technique of using this ligament attachment system is shown in diagram form from FIG. 3 to FIG. 7.

FIG. 3a illustrates the installation of the guide spindles.

FIG. 3b illustrates the production of tunnels 32 from the outside to the inside.

FIG. 4a illustrates the preparation of the housing 44 of the sleeve.

FIG. 4b describes the definitive aspect of the bone tunnels 32 and bone spaces 44.

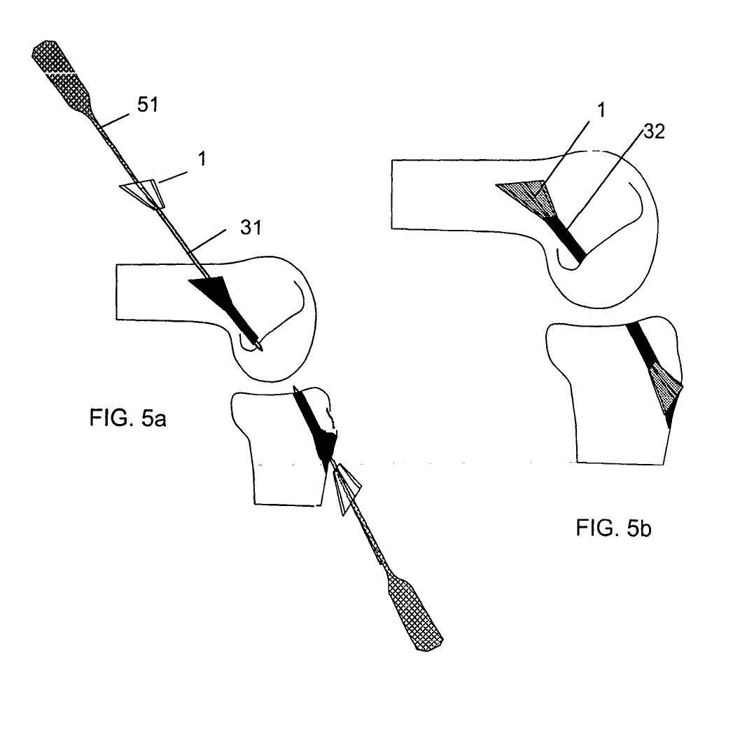

FIG. 5a illustrates the installation of the sleeves 1 by means of a socket holder that also slides over the guide spindles 31.

FIG. 5b illustrates the appearance of the tunnels after installation of sleeves in the femur and in the tibia.

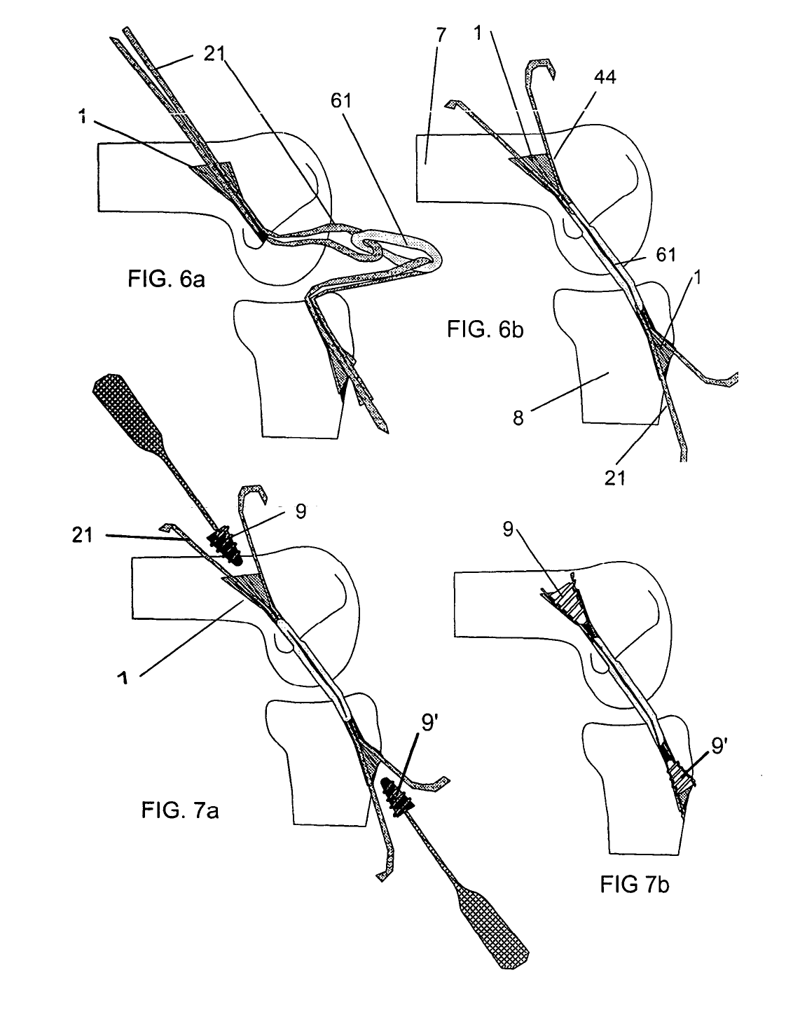

FIG. 6a shows the passage of the strips 21 into the tunnels 32 and the insertion of the graft 61 into the knee by pulling on the strips 21.

FIG. 6b shows the appearance of the graft 61 after its installation.

FIG. 7a illustrates the locking of the graft to the femur.

FIG. 7b shows the appearance of the graft after complete locking.

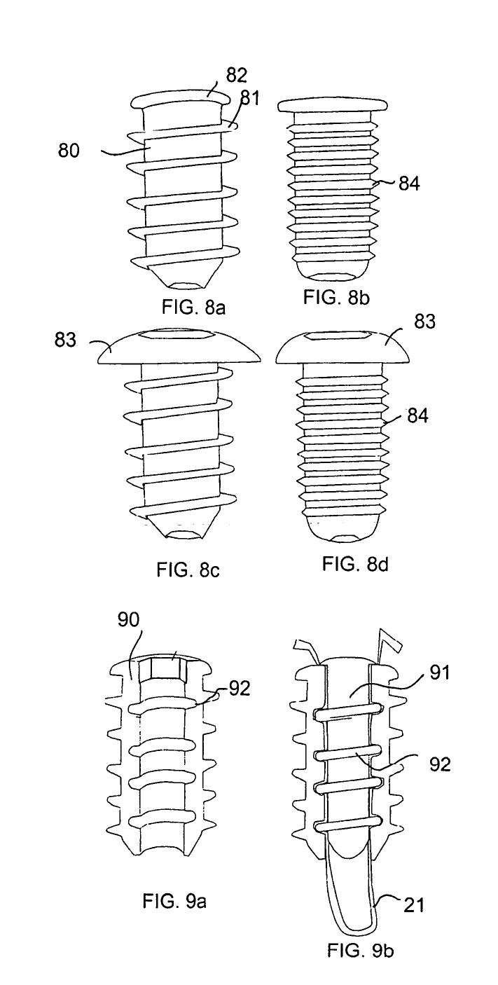

FIGS. 8a and 8b respectively illustrate another embodiment of the invention that consists of a cylindrical sleeve to be screwed down and a cylindrical sleeve to be driven or wedged into the bone tunnel.

FIGS. 8c and 8d illustrate the same cylindrical sleeves but equipped in an optional way with a wide head that is designed to rest on the cortical surface.

FIG. 9a illustrates in longitudinal cutaway a cylindrical sleeve to be screwed down.

FIG. 9b illustrates the same screw after the insertion of the locking element of the strip.

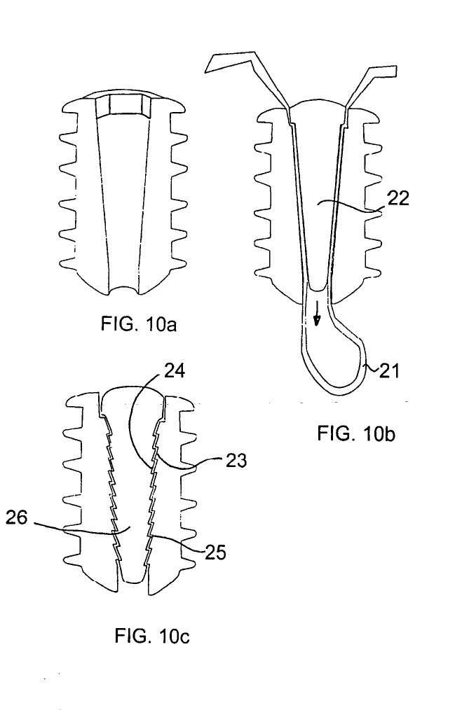

FIGS. 10a-10c illustrate the case where the strip is wedged by a locking element that is not a screw.

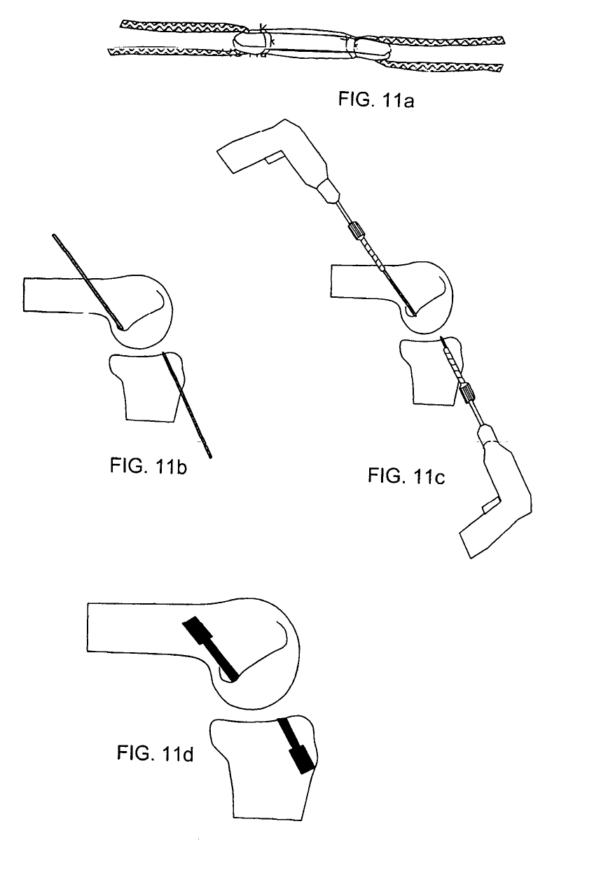

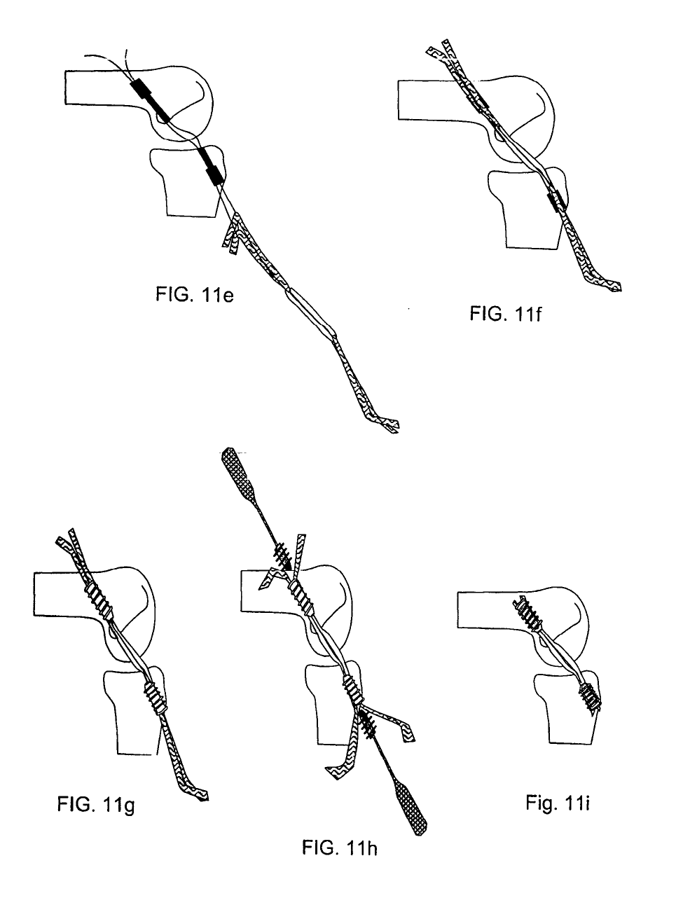

FIGS. 11a-11i illustrate the stages of a process for surgical reconstruction of the anterior cruciate ligament.

### DETAILED DESCRIPTION

FIG. 1a shows diagrammatically and in perspective a sleeve according to the invention that has a first opening 11 and a second, more narrow opening 12 as well as an outside wall 6, and the stopping flange 3. FIGS. 1b and 1c are corresponding horizontal and vertical cutaways that show four anti-rotational flanges 2, the inside wall 6′, and the inside furrow 4′.

FIG. 1d is a longitudinal section that shows the flange 3 and the beveled section of the wide part of the cone, with an angle of approximately 30° between the longitudinal axis b of the sleeve and the plane a that is perpendicular to this axis.

FIG. 1e shows how the sleeve 1 is positioned in a bone tunnel that is made according to 3 different angulations relative to the bone cortex. In each of the cases, the sleeve stops on the cortex where the latter forms an acute angle with the bone tunnel.

FIG. 2 shows the screw 9 in place in the sleeve 1 that compacts the suspension strip 21 of the graft in the furrow 4′ of the wall 6′ of the sleeve 1.

The technique of using this ligament attachment system is shown in diagram form from FIG. 3 to FIG. 7 for a surgical reconstruction of the anterior cruciate ligament in the knee joint.

FIG. 3a illustrates the installation of the guide spindles 31 that are designed to guide the instruments for piercing the bone tunnels to the ends of the femur 7 and the tibia 8.

FIG. 3b illustrates the production of tunnels 32 from the outside to the inside by means of hollow drills that slide on the guide spindles 31. The tunnel is hollowed out all the way through according to a single caliber based on the measurement of the caliber of the ends of the graft.

FIG. 4 illustrates more particularly the preparation of the housing 44 of the sleeve 1 by means of a hollow metal instrument 41 that also slides on the guide spindles 31 and of which one end 42 comprises a conical element with a shape and size that are strictly identical to the definitive sleeve. Thus, this conical element is equipped with cutting edges that prepare the bone furrows that will accommodate the anti-rotation ailerons of the sleeve, and it is also equipped with a cortical stop flange 43 that is just like the definitive sleeve. The penetration is done with a hammer. By pounding, the instrument compacts the walls of the cylindrical tunnel by creating a cone-shaped space whose depth corresponds to the maximum degree of penetration of the instrument, i.e., when its cortical stop flange just abuts against the entrance of the bone tunnel.

FIG. 4b describes the definitive appearance of the bone tunnels 32 and bone spaces 44, whereby the guide spindles are still present.

FIG. 5a illustrates the installation of the sleeves 1 by means of a socket holder that also slides over the guide spindles 31.

The sleeves are pounded in with a hammer until they are locked in their penetration by their conical shape and by the stop 3 of the cortical stop flange.

FIG. 5b illustrates the appearance of the tunnels after installation of the sleeves 1 in the femur and the tibia.

FIG. 6a shows the passage of the strips 21 into the tunnels and insertion of the graft 61 in the knee by pulling on the strips.

FIG. 6b shows the appearance of the graft 61 after its installation.

FIG. 7a illustrates the locking of the graft to the femur by the installation of the locking screw 9, then, tightening the graft 61 in the tibia and locking by a similar screw 9′.

FIG. 7b shows the appearance of the graft after complete locking.

It will be understood that the attachment system as illustrated and described above can comprise considerable advantages:

1: The torque of the screw 9 in its sleeve 1 is known by manufacturing and therefore entirely predictable contrary to the TLS screw whose torque is random and essentially depends on the quality of the receiving bone, highly variable from one individual to the next.

The use of a dynamometric turn screw would even make it possible to finely regulate the torque and make it identical in all patients, regardless of the quality of their bones.

2: The risk of a possible excess insertion depth as noted in the original TLS system is eliminated since, owing to its conical shape, the sleeve stops automatically when it reaches the bottom of its space and when its flange abuts against the cortex at the entrance of the tunnel. The risk of deficient insertion depth also disappears since the sleeve is driven with a hammer until it stops automatically for the reasons already disclosed.

3: The risk of divergence between the screw and the axis of the tunnel (and therefore of the strips), as identified in the TLS system, no longer exists since the conical spaces are produced by means of an instrument that slides on the guide spindles that thus make it possible to align perfectly the axis of the spaces with the axis of the bone tunnels.

The socket holder slides on the same guide spindle that imposes a perfectly controlled direction of the instrument during the installation of the sleeve.

Once the sleeve is in place, the screw has no other option than to regain the prefabricated furrow of the inside wall of the sleeve by automatically ensuring an optimum tightening of the strip.

4: Not only does this system entirely solve all of the residual mechanical problems of the original TLS system while preserving exceptional performance levels, but in addition, it does it using a greatly simplified technique since the bone tunnels are produced integrally according to the caliber of the graft, whereas the TLS system made it necessary to separately produce recessed spaces and fine-caliber tunnels designed for the creation of the housing of the screw.

According to the preferred (but not restrictive) embodiment of this system, the sleeve as well as the screw are produced from biocomposite material, i.e., combining a bioresorbable polymer, for example, of the PLA (polylactic acid) type with an osteo-inductive substance, for example of the TCP (tricalcium phosphate) type. The foreign material that is thus introduced not only is resorbed slowly over time but also it does it by stimulating the local proliferation of bone tissue. After having played their mechanical role, the attachment elements (sleeve and screw) slowly disappear to leave, as it were, room for the bone tissue from the receiving host. The suspension strips can also be manufactured from resorbable material that after complete resorption of the system would leave a perfectly clean and natural environment.

Whereby the biocomposite material is very hard, there are furthermore no longer objections to a reduction if the size of the implants is desired to be based on the situations encountered since the tightening occurs between two elements of equal hardness whereas the original TLS system imposed, as it were, the use of a screw with a large diameter. It is actually by crushing and by compacting the bone around it that the TLS screw makes it possible to obtain an adequate tightening effect of the strip.

Like the TLS screw, this system makes it possible to lock the textile strips as they are used in ligament surgery, but it could also be used as a means for locking simple suture threads which, after any ligament structure has been tightened, could be locked very effectively by tightening between a sleeve and a locking screw, thus eliminating the necessity of making stop knots that are sometimes very difficult to produce.

FIGS. 8 to 10 illustrate a particularly preferred embodiment of the invention. The hollow sleeve in this embodiment is essentially a cylindrical and no longer conical element. In cylindrical mode, it is possible to imagine two types of insertion and anchoring of the hollow element in the bone: either an organ to be screwed down as FIG. 8a shows, or a peg-type element to be driven in (same principle as the sleeve of FIG. 1) as FIG. 8b shows.

The element to be screwed down (FIG. 8a) therefore comprises a cylindrical body 80 of 20 to 25 mm in length for an outside diameter of the cylinder of approximately 10 mm. The outside wall has a wide, relatively sharp thread 81 that resembles a tie screw used in wood or else with a wide and deep thread of spongy-bone screws. This wide and cutting thread 81 achieves an extremely solid bone anchorage. It is also possible to provide to the base a small collar 82 that is designed to stop on the cortex. The oblique insertion of this screw relative to the bone surface would require a small milling of several millimeters so as to increase the penetration of the screw in the bone and to reduce its outer bulk. This small milling would in principle pose no particular technical problem. If so desired, it could also increase the support surface on the cortical bone by replacing the small collar by a true screw head 83 that should be convex and flat as illustrated in FIG. 8c. Of course, such a screw head would also require a small milling to bury it partially in the bone and to reduce the outside bulk.

FIG. 8b shows a cylindrical element that is similar but is designed to be driven into the bone tunnel rather than screwed down. For this purpose, the outside surface area of the cylinder of 10 mm could be equipped with fine stops perpendicular to the large axis of the hollow element and parallel to one another. The cortical support collar would also require a small milling to at least partially bury the head of the peg.

The locking of the strips inside the sleeve can be carried out essentially in two ways:

1—Either by screwing by using the same principle as the original TLS screw. It will be noted, however, that in this device, it is no longer necessary to provide a conical shape to the locking screw since it can be stopped at the entrance of the sleeve.

FIG. 9a shows a cylindrical sleeve 90 to be screwed down in longitudinal cutaway. FIG. 9b shows the same cutaway after insertion of the locking element 91 of the strip 21.

This locking element consists of a screw 91 with a wide pitch and a foam thread (TLS principle) whose diameter is adjusted to wedge the strip by tightening against the inside wall of the sleeve and in the inside milling. This locking screw could have a conical shape like the TLS screw, but this device, as was already said, is no longer actually necessary and a cylindrical section screw would make it possible to obtain the same result, possibly more easily.

2—Or by locking. In this case, the core of the sleeve will have been hollowed out in the shape of a cone and the locking element having the same shape will be simply driven into the conical cavity so as to wedge the strip by the corner effect.

FIG. 10a shows such a sleeve in longitudinal cutaway. FIG. 10b illustrates the attachment mechanism of the strip 21 after insertion of the locking element 22. FIG. 10c constitutes a variant of this device in which the inside wall 23 of the sleeve as well as the outside wall 24 of the locking element 22′ will have been equipped with fine indentations 25 so as to avoid the risk of accidentally locking the system.

An additional advantage of this system is as follows: as soon as the tibial tunnel is made entirely equal to the caliber of the graft, the latter can penetrate into the knee by the tunnel itself from the outside to the inside as is done in the conventional techniques (and no longer through the arthroscopy opening). This makes it possible to protect the remains of the ruptured anterior cruciate ligament, which, it seems, could significantly promote the revascularization of the graft and its incorporation into the bone tissue. It is actually possible to take into consideration that it is from these residual tissues that the vascularization of the graft, which is essential to its incorporation and to its survival, starts up.

The insertion of the graft by the arthroscopy opening as described in the TLS technique of the document of patent WO 2004/045465 requires, on the contrary (and unfortunately), the excision of these tissues so as to prevent their invagination into the tunnel during the insertion of the graft, able to lock the penetration of the graft into its space. One skilled in the art will understand that the latter could therefore prove to be a handicap or a brake to the incorporation and the healing of the graft.

One skilled in the art will understand that the use of cylindrical sleeves makes possible the use of a standard sleeve, for example of caliber 10 mm, which corresponds to the observable maximum caliber for the anterior cruciate ligament grafts. The drilling instrumentation of the tunnel would therefore comprise hollow drills with two segments, a first segment with a variable caliber based on the caliber of the graft (from 6 to 10 mm), and the second segment, with a constant diameter, of 10 mm corresponding to the space of the sleeve. Such a standardization would be more difficult in the case of a conical sleeve because the conical recess of the space is still to be substantially greater than the diameter of the tunnel that receives the graft.

The invention also relates to a new technique for surgical reconstruction of the anterior cruciate ligament by using, for example, a sleeve and a locking element to be screwed down. The method according to the invention is summarized and diagrammed in the following manner with reference to FIGS. 11 a-i:

Preparation of the Graft (FIG. 11a):

A single tendon of the inner hamstring is sampled. The tendon is wound four to five times on itself to obtain a short closed loop with four or five strands. Two transfixion suture points are placed on the two ends of the loop to neutralize the sliding of the strands between one another. A surgical textile strip is run freely through each of the ends of the loop, thus making possible the suspension and the attachment of the ligament loop.

The thus manufactured loop is placed on a table for pulling by means of the strips, and a prestressing of 50 kilos is applied to the system for 15 to 20 minutes before inserting it into the knee. This prestressing deforms the graft somewhat and thus neutralizes any phenomenon of parasitic elongation that is able to occur during the post-surgical period, which would bring about a stress relief in the graft and an at least partial reappearance of the articular laxity. The graft is calibrated so as to know the piercing diameter of the tunnels.

Preparation of the Bone Tunnels (FIGS. 11b-d):

Installation of the guide spindles in the femur and the tibia under arthroscopic monitoring by means of conventional instruments (viewfinders, etc. . . . ). (FIG. 11b) The end of each of the spindles corresponds to the intraarticular anchoring zone that is selected by the surgeon for the docking of the graft.

Piercing of the bone tunnels from the outside to the inside in the femur then in the tibia according to the caliber of the graft (FIG. 11c):

The instrumentation comprises a series of hollow drills with two segments: the distal segment is variable (from 6 to 10 mm) and corresponds to the measured caliber of the graft. The proximal segment is constant and corresponds to the caliber of the sleeve (10 mm). The use of this special drill therefore makes it possible to produce in a single passage the housing of the ligament and that of the sleeve. The cutting is also carried out from the outside to the inside. FIG. 11d diagrammatically shows the appearance of the tunnels after piercing.

Installation and Attachment of the Graft (FIGS. 11e-i):

A pulling thread is inserted into each of the tunnels from the outside to the inside of the knee and then is recovered by the internal anterior arthroscopic approach.

This pulling thread makes it possible to draw the strips into the knee, then through each of the tunnels, and to recover them at the outside opening of each of the tunnels. This method makes it possible to insert the graft via the endoscopic approach by simple pulling on the strips as illustrated in FIGS. 6a and 6b.

A variant consists in inserting a single pulling thread into the femoral tunnel, first of all from the outside to the inside, then to recover this thread through the tibial tunnel from the inside to the outside (FIG. 11e). This thread then makes it possible to draw the strips that suspend the femoral pole of the graft through the tibial tunnel then the knee then through the femoral tunnel (FIG. 11f). This method thus makes it possible to insert the graft through the tibial tunnel as in most of the traditional ligamentoplasty methods.

As was mentioned above, it could be advantageous to the extent where it makes it possible to avoid excessive debridement of the entrance to the tibial tunnel, which could have a negative effect on the subsequent revascularization of the graft.

Passage of the strips into the femoral sleeve and screwing of this sleeve into the bone housing prepared for this purpose (FIG. 11g).

Passage of the strips into the tibial sleeve and screwing of this sleeve into the bone housing prepared for this purpose (FIG. 11g).

Tightening the graft to the femur by simply pulling on the strips. The penetration of the graft in the femur is at a maximum when the latter abuts against the end of the sleeve. Locking of the strips by inserting the locking screw in the femoral sleeve (FIG. 11h).

Tightening of the graft in the tibia by simply pulling on the strips and locking the strips by inserting the locking screw in the tibial sleeve (FIG. 11h).

FIG. 11i shows the final appearance after locking in the femur and in the tibia by the locking screw and section of the strips.

This description relates to an intervention where the process of the sleeve that is described in this report would have been used both for the femur and the tibia. All of the variants are obviously possible, and it will be understood that it is possible to use a hybrid system where the femoral pole of the graft would be attached by an ordinary TLS screw or even an Endobutton-type system and where only the tibial pole would be attached by using the process of the sleeve so as to make it possible to insert the graft through the tibial tunnel.

1. Hollow sleeve (1) to be anchored in a bone tunnel that is designed for the passing of relay strips or suture thread, characterized in that it has an outside wall (6), an inside wall (6′), and two openings (11, 12), whereby said inside wall is able to clamp and lock said strips by the effect of a locking element that is inserted into the sleeve.

2. Sleeve according to claim 1, wherein the outside wall (6) is equipped with a means of connecting to the bone tunnel (32).

3. Sleeve according to claim 2, wherein the means of connection consists of a thread fitted to the bone anchorage.

4. Sleeve according to claim 1, wherein the outside wall is essentially cylindrical in shape, and the inside wall is cylindrical or conical in shape.

5. Sleeve according to claim 4 that comprises in its upper opening a head or a collar that is designed to stop the sleeve on the cortical plane.

6. Sleeve according to claim 1, wherein it is essentially conical in shape and the inside wall is cylindrical or conical in shape.

7. Sleeve according to claim 1, wherein the plane of the entrance opening of the sleeve is sloped by about 20 to 50°, preferably about 30°, relative to a plane that is perpendicular to the large axis of the sleeve.

8. Sleeve according to claim 7, wherein at the junction between said entrance opening and said outside wall (short side) of the sleeve, there is a flange (3) that is designed to abut against the bone cortex, thus keeping the sleeve from penetrating beyond this cortex.

9. Sleeve according to claim 1 that is made of biocomposite material.

10. Sleeve according to claim 1, wherein it is made of a material that is identical to that of the anchoring screw.

11. Sleeve according to claim 2, wherein the outside wall of the sleeve is equipped with at least two, preferably four, anti-rotational ailerons (2).

12. Sleeve according to claim 1, wherein it comprises a threading on the inside wall.

13. Sleeve according to claim 12, wherein the threading is provided in addition to that of the locking element by taking into consideration the thickness of the strips to be locked.

14. Set for attachment device comprising an anchoring element and a sleeve according to claim 1.

15. Set for attachment device according to claim 14, wherein the anchoring element is a screw, a smooth or toothed conical body.

16. Set according to claim 15, wherein the threading is blunt and has a wide pitch, preferably about 5 mm.

17. Set according to claim 14, wherein the distal end of the locking element is rounded and soft.

18. Process for surgical reconstruction by using a graft with strips, wherein at least one sleeve and a suitable locking element, such as a screw, are inserted, whereby said element is designed to lock the strips in the sleeve, itself inserted into at least one previously pierced bone tunnel.

19. Process for surgical reconstruction of the anterior cruciate ligament, wherein a sleeve and a locking element, for example a screw, are used, comprising the following stages:
Preparation of a graft (FIG. 11a), for example from an inner hamstring tendon, wound to obtain a closed loop with several strands
Placements of transfixion suture points at two ends of the loop
Passage of a surgical textile strip through each of the ends of the loop, thus making possible the suspension and the attachment of the ligament loop
Prestressing
Calibration of the graft
Installation of the guide spindles in the femur and the tibia under arthroscopic monitoring to determine the intra-articular anchoring zones (FIG. 11b)
Piercing of bone tunnels from the outside to the inside in the femur then in the tibia with a hollow drill, each time in a single passage, whereby the distal segment is variable and depends on the caliber of the graft, and whereby the proximal segment is constant and corresponds to the caliber of the sleeve
Insertion of a pulling thread into one or each of the tunnels from the outside to the inside to bring the strips into the knee and to recover them at the outside origin of each tunnel
Screwing or locking the sleeves (FIG. 11g)
At the femur, tightening the graft by pulling on the strips until the graft stops at the end of the sleeve, then locking by inserting the locking element or screw
At the tibia, tightening of the graft by pulling on the strips until the graft stops at the end of the tibial sleeve then locking by insertion of the locking element or screw.

20. Process for surgical reconstruction according to claim 19, wherein a single pulling thread is inserted into the femoral tunnel first from the outside to the inside, then recovered through the tibial tunnel from the inside to the outside (FIG. 11e), whereby the thread then makes it possible to draw the strips that suspend the femoral pole of the graft through the tibial tunnel then the knee then through the femoral tunnel (FIG. 11f).
